(19) United States
(12) Patent Application Publication
(10) Pub. No.: US 2026/0124471 A1
(43) Pub. Date: May 7, 2026

(54) PARTICLE BEAM IRRADIATION SYSTEM

(71) Applicants: KABUSHIKI KAISHA TOSHIBA, Kawasaki-shi (JP); TOSHIBA ENERGY SYSTEMS & SOLUTIONS CORPORATION, Kawasaki-shi (JP)

(72) Inventor: Hiromichi RYUTO, Yokohama Kanagawa (JP)

(73) Assignees: KABUSHIKI KAISHA TOSHIBA, Kawasaki-shi (JP); TOSHIBA ENERGY SYSTEMS & SOLUTIONS CORPORATION, Kawasaki-shi (JP)

(21) Appl. No.: 19/427,199

(22) Filed: Dec. 19, 2025

Related U.S. Application Data
(63) Continuation of application No. PCT/JP2024/023927, filed on Jul. 2, 2024.

(30) Foreign Application Priority Data
Jul. 7, 2023 (JP) 2023-112232

(51) Int. Cl. A61N 5/10

(52) U.S. Cl. CPC A61N 5/1081; A61N 2005/1094

(57) ABSTRACT

A particle beam irradiation system includes a first building, a second building different from the first building, an accelerator installed in the first building configured to accelerate a charged particle beam, a beam transport line configured to transport the charged particle beam accelerated by the accelerator, and an irradiation device installed in the second building configured to irradiate an irradiation target with the charged particle beam transported by the beam transport line.

### CROSS-REFERENCE TO RELATED APPLICATIONS

This application is a Continuation Application of PCT/JP2024/023927 filed on Jul. 2, 2024, and the PCT application is based upon and claims the benefit of priority from Japanese Patent Application No. 2023-112232 filed on Jul. 7, 2023, the entire contents of which are incorporated herein by reference.

### BACKGROUND OF THE INVENTION

### Technical Field

The present invention embodiments relate to a particle beam irradiation system.

### Related Arts

Conventionally, particle beam treatment facility including an irradiation device such as a rotating gantry for radiation therapy has been known. To install particle beam treatment facility on a limited site in an urban area, the technique for dividing the hierarchical level of installing a particle accelerator and the hierarchical level of installing an irradiation device in one building into an upper level and a lower level has been known. However, it is necessary to firmly construct the building to install large machines such as a rotating gantry or an accelerator on the upper level located apart from the ground serving as the base of building. In addition, this technique needs to form beam transport lines connected between the upper level and the lower level in complex shapes having many bent portions, which further need to arrange plenty of deflection electromagnets. Accordingly, this technique shall increase the construction cost.

As related arts of the prevent invention, Patent Document 1 through Patent Document 4 disclose particle beam irradiation technology.

Patent Document 1: Japanese U.S. Pat. No. 5,437,527

Patent Document 2: Japanese Patent Application Publication No. 2019-180654

Patent Document 3: Japanese Patent Application Publication No. 2020-779

Patent Document 4: Japanese Patent Application Publication No. 2021-153759

### SUMMARY OF THE INVENTION

The technological problem of the present invention is to efficiently install a particle beam irradiation system on a limited site in an urban area or the like and to contribute to a reduction of construction costs.

According to the present invention embodiment, a particle beam irradiation system includes a first building; a second building different from the first building; an accelerator installed in the first building configured to accelerate a charged particle beam; a beam transport line configured to transport the charged particle beam accelerated by the accelerator; and an irradiation device installed in the second building configured to irradiate an irradiation target with the charged particle beam transported by the beam transport line.

### BRIEF DESCRIPTION OF THE DRAWINGS

FIG. 1 is a plan view showing a particle beam irradiation system according to the first embodiment.

FIG. 2 is a side view showing the particle beam irradiation system according to the first embodiment.

FIG. 3 is a side view showing a half gantry serving as a modification of an irradiation device.

FIG. 4 is a side view showing a particle beam irradiation system according to the second embodiment.

FIG. 5 is a side view showing a particle beam irradiation system according to the third embodiment.

FIG. 6 is a side view showing a slit-type irradiation device serving as an irradiation device according to the third embodiment.

FIG. 7 is a front view showing the slit-type irradiation device serving as the irradiation device according to the third embodiment.

FIG. 8 is a side view showing a beam transport line according to the third embodiment.

FIG. 9 is a side view showing an example of a beam transport line.

FIG. 10 is a side view showing a particle beam irradiation system according to the fourth embodiment.

FIG. 11 is a side view showing a particle beam irradiation system according to the fifth embodiment.

### DETAILED DESCRIPTION OF THE INVENTION

A particle beam irradiation system according to the present invention embodiment includes a first building, a second building different from the first building, an accelerator installed in the first building configured to accelerate a charged particle beam, a beam transport line configured to transport the charged particle beam accelerated by the accelerator, and an irradiation device installed in the second building configured to irradiate an irradiation target with the charged particle beam transported by the beam transport line.

According to the present invention embodiment, it is possible to efficiently install the particle beam irradiation system on a limited site in an urban area or the like and to contribute to a reduction of construction costs.

### First Embodiment

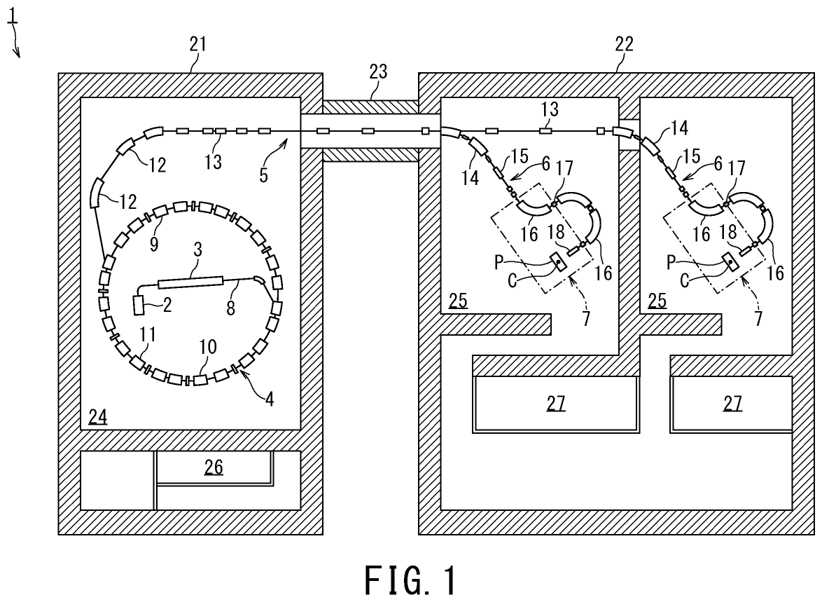

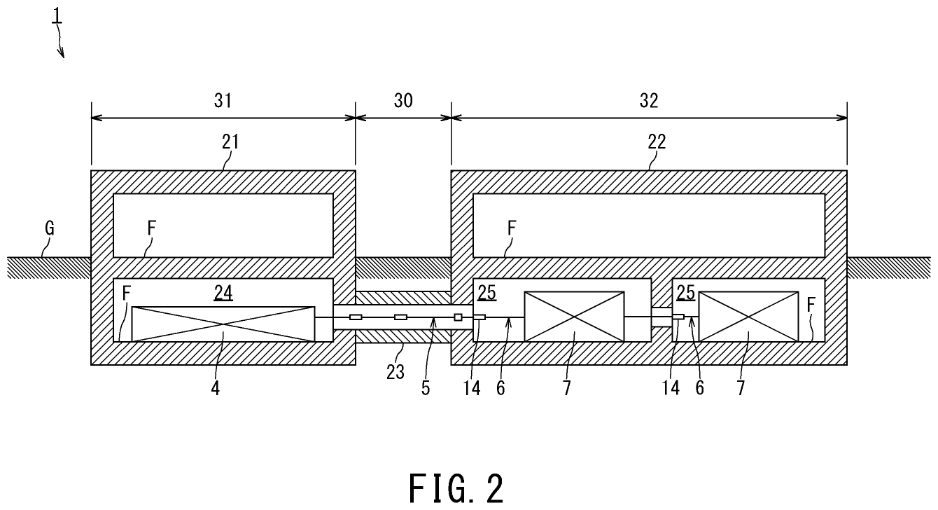

Hereinafter, various embodiments of particle beam irradiation systems will be described in detail with reference to the drawings. First, the first embodiment will be described with reference to FIG. 1 and FIG. 2.

Reference sign 1 in FIG. 1 denotes a particle beam irradiation system according to the first embodiment. The particle beam irradiation system 1 is a particle beam cancer treatment device for treatment by irradiating focal tissues (or cancers) of a patient P as an irradiation target with charged particle beams as radiation of treatment using carbon ions.

A radiation treatment technique using the particle beam irradiation system 1 is referred to as a heavy particles cancer treatment technique. This technique is designed to target cancer focuses (or affected parts) using carbon ions with pinpoint accuracy, thus minimizing damage to normal cells while damaging cancer focuses. In this connection, it is possible to define charged particle beams as heavier beams than electrons in radiation, wherein charged particle beams may include proton beams, heavy particle beams, and the like. Heavy particle beams are defined as heavier beams than helium atoms.

Additionally, the present embodiment describes an example of charged particle beams using carbon, but other examples can be adopted here. For example, it is possible to adopt charged particle beams using helium, oxygen, or neon.

Compared with conventional cancer treatments using X-rays, gamma rays, or proton beams, cancer treatments using heavy particle beams with high performance of killing cancer focuses are characterized by a low dose of radiation on the surface of the body of the patient P and a peak dose of radiation at cancer focuses. Accordingly, it is possible to reduce the number of times of irradiation and side effects, thus shortening the period of treatment furthermore.

For example, charged particle beams may be reduced in velocity due to loss of kinetic energy when passing through the body of the patient P, while resistance, which may be changed approximately inversely proportional to the square of velocity, will be exerted on charged particle beams, and therefore charged particle beams will be rapidly stopped when reaching a certain level of velocity. The point of stopping charged particle beam is called the Bragg peak at which high energy will be emitted therefrom. The particle beam irradiation system 1 is designed to adjust the Bragg peak to the position of focal tissues (or affected parts) of the patient P, thus killing focal tissues alone while suppressing damage to normal tissues.

The particle beam irradiation system 1 includes an ion generator 2, a linear accelerator 3, a circular accelerator 4, a main transport line 5, a sub-transport line 6, and a rotating gantry 7. Additionally, the main transport line 5 and the sub-transport line 6 are combined to form beam transport lines.

The ion generator 2 having an ion source of carbon ions serving as charged particles is configured to generate charged particle beams using carbon ions. The linear accelerator 3 having a linear shape in a plan view is configured to accelerate ions generated by the ion generator 2 into charged particle beams. The linear accelerator 3 is configured to introduce charged particle beams into the circular accelerator 4.

The circular accelerator 4 having a ring shape in a plan view is configured to further accelerate charged particle beams. Here, charged particle beams will accelerate to approximately 70% of the speed of light while circulating the circular accelerator 4 about one million times. Then, charged particle beams accelerated by the circular accelerator 4 will be transported to the rotating gantry 7 by way of the main transport line 5 and the sub-transport line 6. The patient P to be irradiated with charged particle beam is placed inside the rotating gantry 7. An isocenter C, which is a position to be irradiated with charged particle beams in the most-concentrated manner, is set inside the rotating gantry 7, and therefore affected parts of the patient P are positioned at the isocenter C.

All the ion generator 2, the linear accelerator 3, the circular accelerator 4, the main transport line 5, and the sub-transport line 6, the inside of which is in a vacuum, include vacuum ducts 8 (or beam pipes) that extend in an integrated manner. Charged particle beams shall propagate through the vacuum ducts 8. The vacuum ducts 8 are used to form a transport path to transport charged particle beams from the ion generator 2 to the rotating gantry 7. That is, the vacuum ducts 8 are of a sealed continuous space with a sufficient degree of vacuum to pass charged particle beams therethrough.

The circular accelerator 4 includes high-frequency acceleration cavities 9, deflection electromagnets 10, and convergence electromagnets 11. The high-frequency acceleration cavity 9 is configured to accelerate carbon ions by controlling the frequency of a magnetic field and an accelerating electric field.

The main transport line 5 includes deflection electromagnets 12 and convergence electromagnets 13. The main transport line 5 extends from the circular accelerator 4. A plurality of sub-transport lines 6 is connected to the linear portion of the main transport line 5.

Each sub-transport line 6 includes deflection electromagnets 14 and convergence electromagnets 15. In the present embodiment, two sub-transport lines 6 are connected to one main transport line 5. Each sub-transport line 6 extends to the rotating gantry 7.

That is, the beam transport line formed by the main transport line 5 and a plurality of sub-transport lines 6 is used to transport charged particle beams accelerated by the circular accelerator 4 to the rotating gantry 7.

The rotating gantry 7 is a device having a cylindrical shape, details of which are omitted in illustration. The rotating gantry 7 is installed in such a manner that the axis of a cylinder will be directed in the horizontal direction. The rotating gantry 7 can rotate in the entire circumference about the horizontal axis. The rotating gantry 7 corresponds to an irradiation device according to the first embodiment.

End rings (not shown here) are fixed at the front edge and the rear edge of the rotating gantry 7. Rotation drive units (not shown here), which are configured to support the end rings in a rotary manner and equipped with drive motors, are arranged below the end rings. The rotation drive units are supported on the frame. The driving force of the rotation drive units is applied to the rotating gantry 7 via the end rings, and therefore the rotating gantry 7 shall rotate around the horizontal axis.

In addition, the rotating gantry 7 includes deflection electromagnets 16, convergence electromagnets 17, and an irradiation nozzle 18. The irradiation nozzle 18, the deflection electromagnets 16, and the convergence electromagnets 17 are supported by the rotating gantry 7 such that they can rotate together with the rotating gantry 7.

The rotating gantry 7 is equipped with the vacuum duct 8 continuously running from the sub-transport line 6. First, the vacuum duct 8 connects from the end portion of the rotating gantry 7 to the inside thereof along the horizontal axis. The vacuum duct 8 extends outwardly from the outer circumferential surface of the rotating gantry 7 and then extends again toward the inside of the rotating gantry 7. The irradiation nozzle 18, at which the distal end of the vacuum duct 8 is placed, extends to the close position of the patient P.

A predetermined rotation mechanism (not shown here) is attached to part of the vacuum duct 8 extending along the horizontal axis of the rotating gantry 7. Part of the vacuum duct 8 outside the rotation mechanism is in stationary condition while another part of the vacuum duct inside the rotation mechanism may rotate in rotation of the rotating gantry 7.

The irradiation nozzle 18 is attached to the distal end of the vacuum duct 8 and configured to irradiate the patient P with charged particle beams transported by the deflection electromagnets 16 and the convergence electromagnets 17. The irradiation nozzle 18 is fixed onto the inner circumferential surface of the rotating gantry 7. In this connection, the irradiation nozzle 18 is adjusted to irradiate charged particle beams in a perpendicular direction to the horizontal axis.

By rotating the rotating gantry 7 under the condition that patient P is at a position of the horizontal axis, it is possible to rotate the irradiation nozzle 18 around the patient P in stationary state. For example, it is possible to rotate the irradiation nozzle 18 around the patient P (at the horizontal axis) by an arbitrary total angle within 360 degrees in rotation of 180 degrees in one way and another way along the circumferential direction of the rotating gantry 7. Subsequently, it is possible to irradiate charged particle beams in any direction around the patient P. That is, the rotating gantry 7 is a device capable of changing the radiation direction of charged particle beams transported by the sub-transport line 6 with respect to the patient P. Accordingly, it is possible to accurately irradiate affected parts of patient P in an optimal direction while reducing any burden on patient P.

The irradiation nozzle 18 can be moved around the isocenter C, at which the patient P as an irradiation target is placed, while keeping equal distances apart from the isocenter C along the entire circumference thereof, wherein the irradiation nozzle 18 can change the irradiation direction of charged particle beams toward the isocenter C.

The irradiation nozzle 18 includes appliances such as a scanning electromagnet, a beam monitor, and an energy modulator, detailed illustrations of which are omitted here. The scanning electromagnet is configured to adjust the amount of current flowing therethrough and the direction of current and to thereby achieve fine adjustment in the propagating direction of charged particle beams emitted from the irradiation nozzle 18, thus conducting scanning using charged particle beams in a relatively small range of areas. The beam monitor is configured to monitor charged particle beams and to measure a dose of beams, an irradiated position, and a flatness of beams in fields. The energy modulator is configured to adjust energy of charged particle beams, thus adjusting the depth of body in which charged particle beams can reach the inside of the body of the patient P. For example, the energy modulator is configured with a range modulator, a scatterer, a ridge filter, a collimator, a bolus, an applicator, or any combination thereof.

In this connection, the deflection electromagnets 10, 12, 14, 16 and the convergence electromagnets 11, 13, 15, 17 described above are configured with electromagnets for generating a magnetic field to form a transport pathway of charged particle beams, which are arranged to encompass the outer circumference of the vacuum ducts 8. The deflection electromagnets 10, 12, 14, 16 are configured to change the propagating direction of charged particle beams along the vacuum ducts 8. The convergence electromagnets 11, 13, 15, 17 are configured to control convergence and divergence of charged particle beams. In this connection, the convergence electromagnets 11, 13, 15, 17 are configured with quadrupole electromagnets, sextupole electromagnets, or the like.

In the present embodiment, the deflection electromagnets 10, 12, 14, 16 and the convergence electromagnets 11, 13, 15, 17 can be configured with superconducting electromagnets.

As shown in FIG. 1 and FIG. 2, the particle beam irradiation system 1 according to the first embodiment includes a first building 21, a second building 22 different from the first building 21, and a connecting passage 23 connecting these buildings. The first building 21 and the second building 22 are architectures having hierarchical structures. For example, each of the first building 21 and the second building 22 has a first floor, whose floor F is established at a higher position than ground G, and a first basement whose floor F is established at a lower position than ground G. In this connection, the first building 21 and the second building 22 have their first basements whose floors F are established at the same position.

An accelerator room 24 for installing machinery such as the linear accelerator 3 and the circular accelerator 4 therein is built on the first basement of the first building 21. An irradiation room 25 for installing machinery such as the rotating gantry 7 is built on the first basement of the second building 22.

FIG. 1 and FIG. 2 show simplified illustrations for prompting understanding, wherein the actual form of the rotating gantry 7 is regarded as a large device. For this reason, compared with the accelerator room 24 for installing the circular accelerator 4 therein, the irradiation room 25 for installing the rotating gantry 7 therein may have the floor F lowered in level and a highly raised ceiling.

In addition, the first building 21 may include other areas than the accelerator room 24, for example, an accelerator control room 26 accommodating staff members who can control the linear accelerator 3 and the circular accelerator 4 shall be constructed in the first building 21. The second building 22 may include other areas than the irradiation room 25, for example, an irradiation control room 27 accommodating staff members who can control the rotating gantry 7 will be constructed in the second building 22.

The first building 21 and the second building 22 are connected through the connecting passage 23 that is built underground. The main transport line 5 extends from the accelerator room 24 to the irradiation room 25 through the connecting passage 23. A passage, which is not shown but through which any person can move between the first building 21 and the second building 22, can be built separately from the connecting passage 23 between the first building 21 and the second building 22.

The interior spaces of the first building 21 and the second building 22 are each divided into a radiation control area to install the accelerator room 24 and the irradiation room 25 and a normal area other than the radiation control area. The radiation control area is provided to avoid unnecessary exposure to radiation by any person and defined as an area that is clearly classified as a place exposed to a certain dose of radiation or more and that is provided to prevent unnecessary entry of any person. Construction of radiation control areas is stipulated by law. In this connection, the connecting passage 23 may serve as part of the radiation control area.

All the linear accelerator 3, the circular accelerator 4, the main transport line 5, the sub-transport line 6, and the rotating gantry 7 are regarded as machinery configured to emit radioactive rays during operation, which are therefore installed in the radiation control area. The radiation control area is partitioned by a shielding wall used to shield radiation. The normal area is partitioned by a normal wall that is not intended to shield radiation.

Generally, shielding walls have thickness T of 1-2 meters or more. When shielding walls include metal plates made of lead or iron therein, it is possible to reduce the thickness T of shielding walls to 1 meter or less. Herein, shielding walls include ceilings and floors of radiation control areas.

Any designer who is designing the particle beam irradiation system 1 may be inclined to locate the linear portions of beam transport lines at the position connecting the first building 21 and the second building 22. As shown in FIG. 1, for example, the main transport line 5 has a linear portion connected between the first building 21 and the second building 22. That is, part of the main transport line 5 disposed in the connecting passage 23 has a straight shape. This may allow a reduction of the thickness of the shielding wall to avoid radiation around the main transport line 5, thus reducing construction costs.

As shown in FIG. 2, the connecting passage 23 is built underground. That is, part of the main transport line 5, which is used to connect the first building 21 and the second building 22, is built underground. This may prevent leakage of radiation from the main transport line 5 to the outdoors.

In the first embodiment, the first building 21 is built on one of two building sites 31, 32 adjoining together across a road site 30, while the second building 22 is built on the other of two building sites 31, 32. This may allow the construction of the particle beam irradiation system 1 while maintaining roads used for any person or any vehicle to travel along. For example, the particle beam irradiation system 1 can be built in a certain place having a limited acquisition of land such as an urban area. The road site 30 includes any site used as sidewalks, roadways, railways (or railway tracks), and water channels (e.g., waterway, canals). The roadway includes outdoor roadways, tunnels, and underground roads.

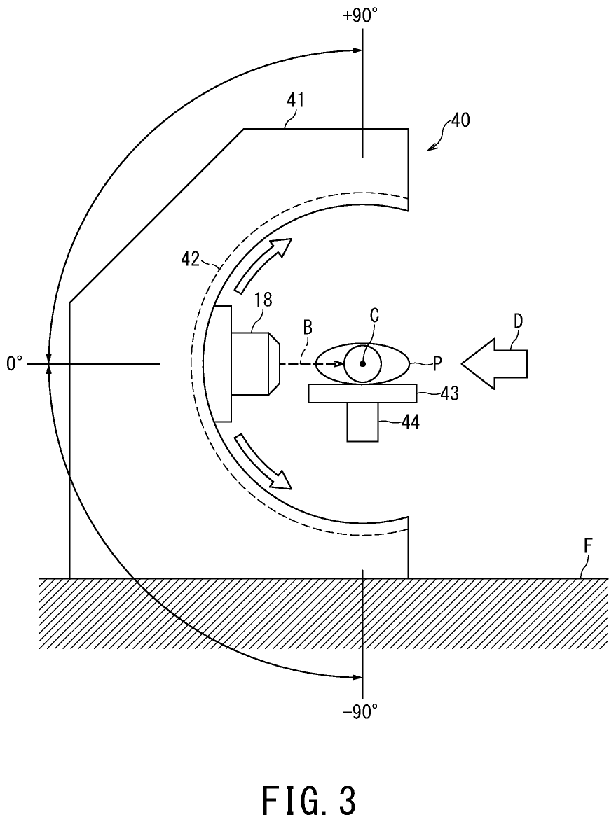

Next, a modification will be described with reference to FIG. 3. In the foregoing embodiment, the rotating gantry 7 exemplifies an irradiation device, but in the modification, a half-gantry 40 of a semi-rotary type exemplifies an irradiation device. The following descriptions refer to the front side (or the forward side) of the half-gantry 40 as shown on the right side of the paper of FIG. 3.

The half-gantry 40 is equipped with the irradiation nozzle 18 configured to change the radiation direction of a charged particle beam B directed to the isocenter C. The irradiation nozzle 18 can be moved around the isocenter C to place the patient P serving as an irradiation target at equal distances apart from the isocenter C in the circumferential direction. The range of movement is 240 degrees or less. That is, the half-gantry 40 indicates an irradiation device whose range of rotation is two thirds or less (i.e., 240 degrees or less) of the entire circumference. The half-gantry 40 is smaller in size than a rotary-type gantry in full circumference.

The irradiation nozzle 18 of the half-gantry 40 according to the modification is set to have a range of movement of 180 degrees in the circumferential direction around the isocenter C. If the position of the irradiation nozzle 18 is at zero degrees when emitting the charged particle beam B in the horizontal direction, for example, the irradiation nozzle 18 can be moved in a range from +90 degrees to −90 degrees. Due to the range of rotation by 90 degrees in one way and another way along the circumferential direction, the irradiation nozzle 18 can be rotated by any angle within 180 degrees in total. For example, the irradiation nozzle 18 can be moved along a C-shape rail 42 having a C-shape in side view, which is attached to a housing 41 of the half-gantry 40.

It is possible to change the radiation direction of the charged particle beam B directed to the isocenter C within the range of movement of the irradiation nozzle 18. When the irradiation nozzle 18 is at the position of +90 degrees, for example, the charged particle beam B is emitted directly from above the patient P. In addition, when the irradiation nozzle 18 is at the position of −90 degrees, the charged particle beam B is emitted directly from below the patient P.

The patient P is placed on a movable table 43. The movable table 43 supported by a movable arm 44 will be moved while carrying the patient P thereon, thus locating the affected part of the patient P at the isocenter C. Due to the movement of the movable table 43, it is possible to adjust the positioning by moving the patient P to the irradiation position of the charged particle beam B. Accordingly, it is possible to irradiate focal tissues of the patient P with the charged particle beam B with optimal accuracy.

The movable table 43 ensures that the patient P can enter an opening part of the half-gantry 40 and be placed at the isocenter C. For example, the movable table 43 carrying the patient P can enter in the front side direction (or the direction of arrow D in FIG. 3) of the half-gantry 40. This allows the patient P to be placed at the isocenter C by entering in an appropriate direction.

In the first embodiment, the first building 21 for installing machinery such as the linear accelerator 3 and the circular accelerator 4 and the second building 22 for installing machinery such as the rotating gantry 7 and the half-gantry 40 are constructed separately. This may allow the particle beam irradiation system 1 to be efficiently built on a limited site in an urban area, thus contributing to a reduction of construction costs.

### Second Embodiment

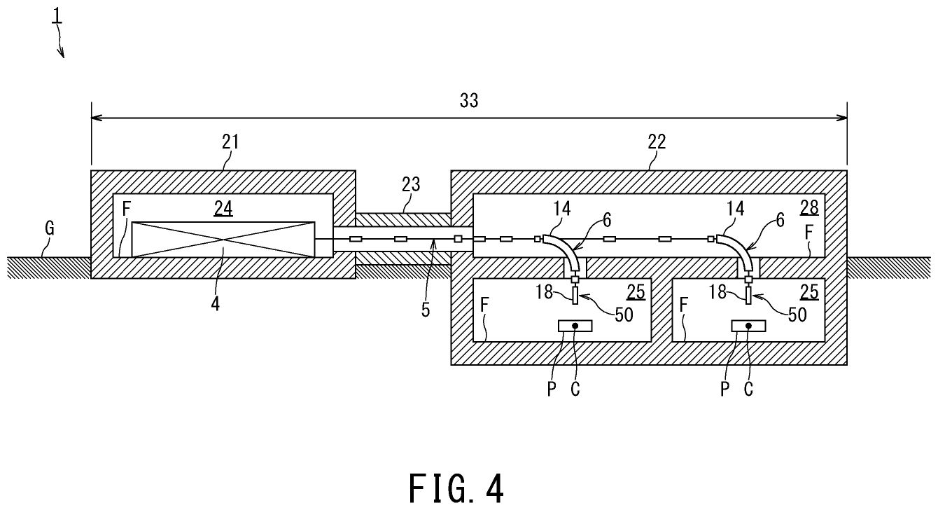

Next, the second embodiment will be described with reference to FIG. 4. In this connection, the same constituent parts as the constituent parts described in the foregoing embodiment will be denoted by the same reference signs; hence, duplicate descriptions will be omitted here.

In the second embodiment, a stationary irradiation device 50 exemplifies an irradiation device. The stationary irradiation device 50 is set to a fixed angle of emitting charged particle beams toward the isocenter C at which the patient P serving as an irradiation target is placed. For example, the stationary irradiation device 50 configured to emit charged particle beams toward the patient P in the vertical direction at an angle other than the horizontal direction is installed in the irradiation room 25.

In the second embodiment, the first building 21, the second building 22, and the connecting passage 23 are built on one building site 33. The first building 21 has a first floor above ground. The second building 22 has a first floor above ground and a first basement below ground. The connecting passage 23 is built on the ground.

The accelerator room 24 to install machinery such as the circular accelerator 4 is built on the first floor of the first building 21. The irradiation room 25 to install the stationary irradiation device 50 is built in the first basement of the second building 22. That is, the level of the accelerator room 24 differs from the level of the irradiation room 25, and therefore the floor F of the accelerator room 24 and the floor F of the irradiation room 25 differ from each other in height. In this connection, the accelerator room 24 can be built in the first basement, while the irradiation room 25 can be built in the second basement.

The main transport line 5 extends from the accelerator room 24 in the first building 21 to a predetermined room 28 on the first floor of the second building 22 through the connecting passage 23. The sub-transport line 6 is bent downwardly from the room 28 and extends to the irradiation room 25 in the first basement of the second building 22. The trajectory of charged particle beams is bent downwardly by the deflection electromagnets 14 of the sub-transport line 6.

In the second embodiment, it is possible to reduce the number of defection electromagnets 14 when the sub-transport line 6 emits charged particle beams to the stationary irradiation device 50 in the vertical direction or in the oblique direction. When the level of the accelerator room 24 is identical to the level of the irradiation room 25, it is necessary to turn the trajectory of charged particle beams, which are emitted from the circular accelerator 4 in the horizontal direction, in the upward direction temporarily, and to turn the trajectory in the horizontal direction, and then to turn the trajectory in the downward direction. That is, it is necessary to provide at least three deflection electromagnets 14. However, it is sufficient for the second embodiment to provide only one deflection electromagnet 14 for turning the trajectory of charged particle beams, which are emitted from the circular accelerator 4 in the horizontal direction, in the downward direction.

### Third Embodiment

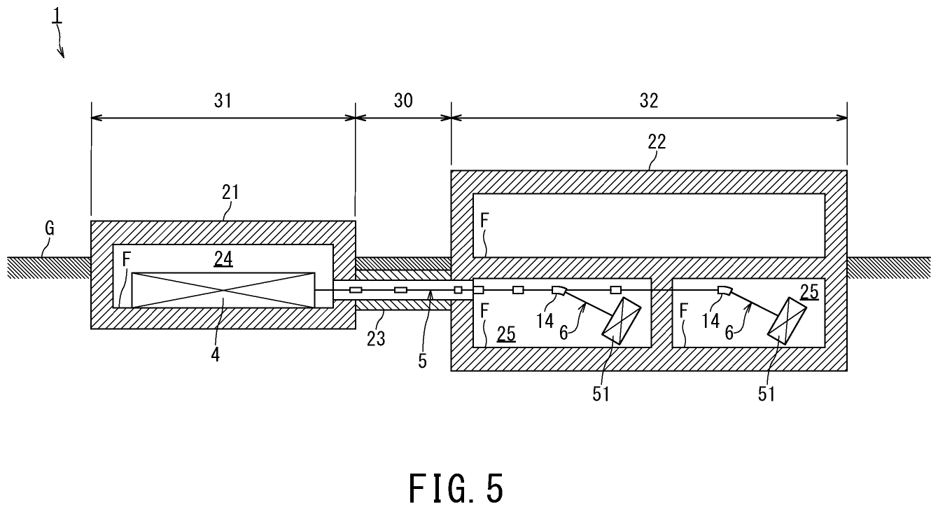

Next, the third embodiment will be described with reference to FIG. 5 through FIG. 8. In this connection, the same constituent parts as the constituent parts described in the foregoing embodiments will be denoted by the same reference signs; hence, duplicate descriptions will be omitted here.

As shown in FIG. 5, the third embodiment refers to a slit-type irradiation device 51 exemplifying an irradiation device. The first building 21 and the second building 22 are built on one side or another side of two building sites 31, 32 adjoining each other across the road site 30. The first building 21 has a first basement below ground. The second building 22 has a first floor above ground and a first basement below ground. In addition, the connecting passage 23 is built underground.

The accelerator room 24 for installing machinery such as the circular accelerator 4 is built in the first basement of the first building 21. The irradiation room 25 for installing the slit-type irradiation device 51 is built in the first basement of the second building 22.

The first basement of the first building 21 and the first basement of the second building 22, both having the same level, are built at different depths, and therefore the floor F of the accelerator room 24 and the floor F of the irradiation room 25 differ from each other in height. For example, the floor F of the accelerator room 24 is set at a higher position than the floor F of the irradiation room 25. That is, the slit-type irradiation device 51 is installed at a lower position than the circular accelerator 4.

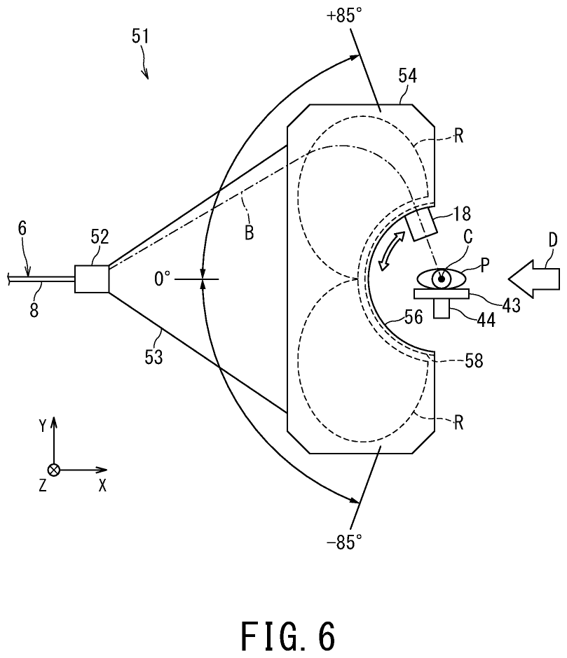

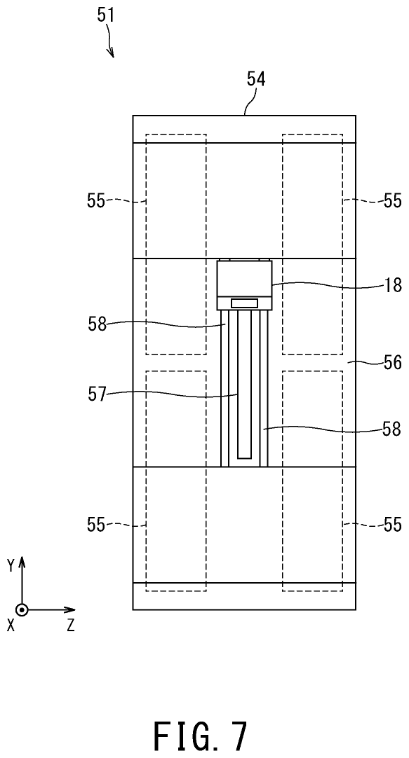

The slit-type irradiation device 51 will be described with reference to FIG. 6 and FIG. 7. The following descriptions refer to the front side (or the forward side) of the split-type irradiation device 51 as shown on the right side of the paper of FIG. 6. Assuming an X direction as the extending direction of the vacuum duct 8 of the sub-transport line 6 and the emitting direction of the charged particle beam B, a Y direction is assumed as an up-down direction of the paper of FIG. 6 perpendicular to the X direction, while a Z direction is assumed to be perpendicular to the X and Y directions.

A deflection electromagnet 52 is attached to the end portion of the vacuum duct 8 of the sub-transport line 6. An expansion duct 53 spreading in a triangular shape (or a fan shape) in the side view of the deflection electromagnet 52 is attached to the deflection electromagnet 52. The expansion duct 53 spreads in the Y direction from the end portion of the vacuum duct 8 of the sub-transport line 6. A main body 54 is connected to the distal end of the expansion duct 53. The main body 54 has a rectangular shape vertically elongated in the side view. The interior space of the expansion duct 53 and the interior space of the main body 54 are connected as a tightly sealed space with a degree of vacuum in succession with the vacuum duct 8 of the sub-transport line 6.

Plenty of deflection electromagnets 55 (FIG. 7), which are configured to deflect charged particle beams B input thereto in a wide range of angles and to thereby converge charged particle beams B at the isocenter C, are arranged inside the main body 54. The deflection electromagnets 55 shall produce effective magnetic field regions R (FIG. 6).

For example, a pair of deflection electromagnets 55, which are paired in the Z direction, is arranged inside the main body 54. Two pairs of deflection electromagnets 55 are arranged to adjoin in the Y direction. One pair of deflection electromagnets 55 can generate one effective magnetic field region R. In the example of FIG. 6, two pairs of deflection electromagnets 55 vertically adjoining together can generate two effective magnetic field regions R vertically adjoining together.

The effective magnetic field region R is generated in a crescent-moon-like shape (or a crescent shape) in the side view. It is possible to control the trajectory of charged particle beam B by controlling the strength of the effective magnetic field region R. Therefore, it is possible to emit the charged particle beam B at an arbitrary angle around the isocenter C. Assuming the inclination of the reference trajectory, which does not deflect the trajectory of charged particle beam 8, at zero degrees, for example, it is possible to change the irradiation angle of the charged particle beam B within a range between +85 degrees and −85 degrees around the isocenter C.

In this connection, the reference trajectory is defined as a trajectory of charged particle beam B emitted straightly to the isocenter C from the vacuum duct 8.

In the example of FIG. 6, two effective magnetic field regions R vertically adjoining together have the same shape and the same strength. That is, two effective magnetic field regions R will be formed in a vertically symmetrical manner, but in another manner adopted. For example, two effective magnetic field regions R can be formed in a vertically asymmetrical manner. That is, two effective magnetic field regions R vertically adjoining together can be formed differently in shape and in strength. Alternatively, a single effective magnetic field region R can be formed on one of upper and lower sides. The center in the range of angles of charged particle beams B varied in the circumferential direction around the isocenter C can be deviated from the reference trajectory of charged particle beams B.

The front side of the main body 54 has a recess 56 caved in a semicircular shape in the side view. The patient P is placed at the recess 56. The movable table 43 can move the patient P to enter the recess 56 on the front side of the main body 54 and to place the patient P at the isocenter C. For example, the movable table 43 carrying the patient P can be moved to enter in the direction of the front side of the slit-type irradiation device 51 (i.e., the direction of arrow D in FIG. 6). This may allow the patient P to enter in an appropriate direction, thus placing the patient P at the isocenter C.

A slit 57 extending in the circumferential direction around the isocenter C, at which the patient P serving as an irradiation target is placed, is formed on the front side of the main body 54. For example, the slit 57 (FIG. 7) is formed in a vertically elongated shape. The slit-type irradiation device 51 is configured to emit the charged particle beam B at an arbitrary angle from the slit 57 toward the isocenter C. In this connection, the slit 57 is closed with an extremely heat-resistant and extremely cold-resistant polyimide film, wherein the slit 57 is formed in the state of capacitating the charged particle beam B to propagate therethrough while maintaining the vacuum condition inside the main body 54.

The slit-type irradiation device 51 is equipped with the irradiation nozzle 18 configured to change the irradiation direction of the charged particle beam B with respect to the isocenter C. The irradiation nozzle 18 can be moved in the circumferential direction around the isocenter C, at which the patient P serving as an irradiation target will be placed, at equal distances apart from the isocenter C. The range of movement should be 180 degrees or less, for example, 170 degrees or less.

When the reference trajectory of the charged particle beam B is tilted at zero degrees, for example, the irradiation nozzle 18 can be moved within a range of +85 degrees to −85 degrees. The irradiation nozzle 18 can be rotated by 85 degrees in one way and another way in the circumferential direction; hence, the irradiation nozzle 18 can be rotated by an arbitrary angle within 170 degrees in total. For example, the irradiation nozzle 18 can be moved along a C-shape rail 58, which is formed in the C-shape in the side view, and which is arranged in the recess 56 of the main body 54 of the slit-type irradiation device 51.

The irradiation nozzle 18 may move along the shape (or the shape of boundary) of an emission side of the effective magnetic field region R in the side view. The charged particle beam B, which is propagating from the emission side of the effective magnetic field region R toward the isocenter C, should pass through the irradiation nozzle 18, which will finely adjust the propagation of the charged particle beam B.

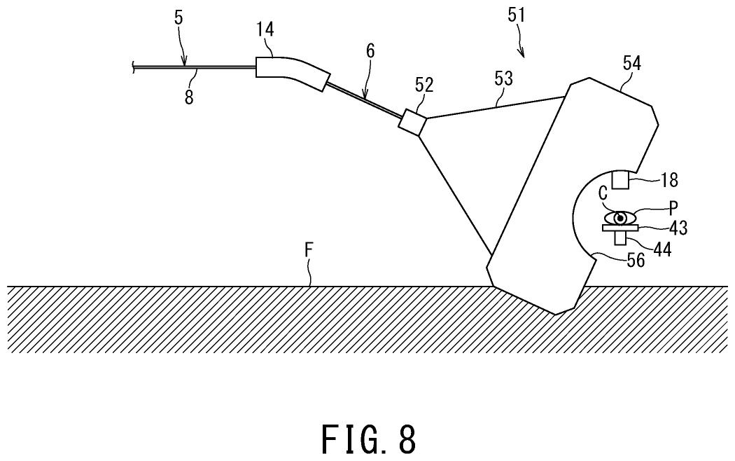

To facilitate understanding, FIG. 6 and FIG. 7 show the situation in which the X direction of the slit-type irradiation device 51 is coordinated in conformity with the horizontal direction. To actually install the slit-type irradiation device 51, for example, the entirety of the slit-type irradiation device 51 should be tilted as shown in FIG. 8. For example, the slit-type irradiation device 51 is installed on the floor F by tilting the longitudinal direction (or the Y direction) of the main body 54.

In the third embodiment, the upper part of the main body 54 is tilted to face the patient P. Although the irradiation range of the charged particle beam B is set to a range of 170 degrees around the isocenter C, it is possible to emit the charged particle beam B to the patient P directly from the above due to the tilt of the slit-type irradiation device 51.

That is, the slit-type irradiation device 51 is installed in the tilted state such that the reference trajectory of the charged particle beam B undeflected by the slit-type irradiation device 51 will be tilted against the horizontal direction (or the horizontal axis). This may result in a practical range of angles at which the charged particle beam B will be emitted to the patient P serving as an irradiation target.

In addition, since the slit-type irradiation device 51 is installed on the floor F such that the longitudinal direction thereof is tilted against the floor F of the irradiation room 25, it is possible to lower the height of the ceiling secured necessary for the installation of the slit-type irradiation device 51.

In the third embodiment, the tilted angle of the reference trajectory (i.e., the X axis) of the charged particle beam B against the horizontal axis is set at 25 degrees. This angle shall be ranged between 20 degrees and 90 degrees.

In the tilted state of the slit-type irradiation device 51, as shown in FIG. 5, the floor F of the irradiation room 25 is built at a lower position than the floor F of the accelerator room 24. This may reduce the number of deflection electromagnets 14 in the sub-transport line 6 when emitting the charged particle beam B into the slit-type irradiation device 51 from the sub-transport line 6 in an oblique direction (or a vertical direction).

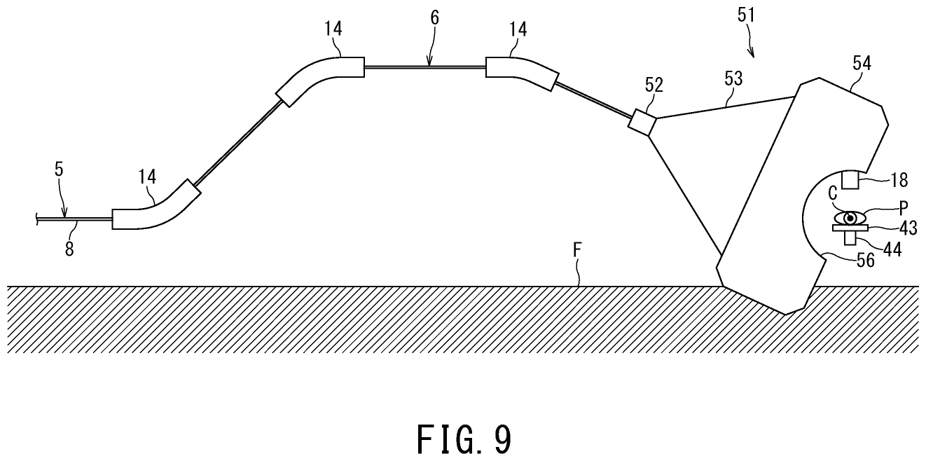

FIG. 9 shows the main transport line 5 and the sub-transport line 6 regarded as the conventional example (or the comparative example) of beam transport lines. In the conventional example, the slit-type irradiation device 51 is installed on floor in a tilted manner on the presupposition that the floor F of the accelerator room 24 and the floor F of the irradiation room 25 are set at the same height. In this case, the vacuum duct 8 extending from the main transport line 5 should be extended in the horizontal direction, temporarily bent obliquely upwards by the sub-transport line 6, extended in the horizontal direction, and then bent obliquely downwards. For this reason, FIG. 9 shows an example of the sub-transport line 6 necessarily including three deflection electromagnets 14.

In contrast, in the third embodiment, as shown in FIG. 8, the vacuum duct 8 extending from the main transport line 5 should be extended in the horizontal direction, and subsequently bent obliquely downwards by the sub-transport line 6 near the slit-type irradiation device 51. That is, it is sufficient for the sub-transport line 6 to install only one deflection electromagnet 14 therein.

The third embodiment is designed to tilt the upper part of the main body 54 of the slit-type irradiation device 51 toward the patient P; however, it is possible to tilt the upper part of the main body 54 to be separated from the patient P. In addition, it is possible to use the slit-type irradiation device 51 without any tilt.

### Fourth Embodiment

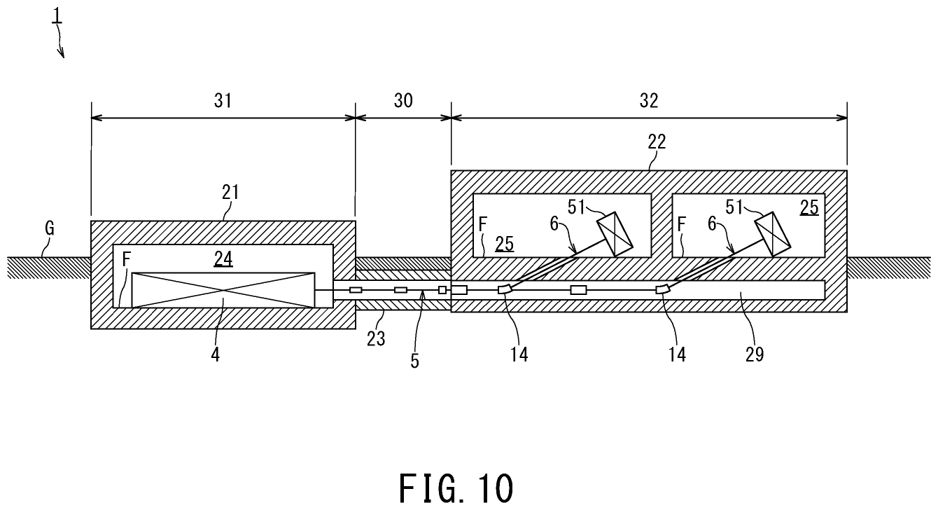

Next, the fourth embodiment will be described with reference to FIG. 10. In this connection, the same constituent elements as the constituent elements described in the foregoing embodiments will be denoted by the same reference signs; hence, duplicate descriptions thereof will be omitted here.

In the fourth embodiment, the slit-type irradiation device 51 exemplifies an irradiation device. The slit-type irradiation device 51 is installed on floor such that the entirety thereof is tilted. Contrary to the third embodiment (FIG. 8) described above, the fourth embodiment is designed to tilt the upper part of the main body 54 to be separated from the patient P.

According to the fourth embodiment, the first building 21 and the second building 22 are built in one site and another site in two building sites 31, 32 adjoining each other across the road site 30. The first building 21 has a first basement below ground. The second building 22 has a first floor above ground. In addition, the connecting passage 23 is built underground. An underground channel 29 continuing from the connecting passage 23 is built on the underground of the second building 22.

The accelerator room 24 to install machinery such as the circular accelerator 4 is built in the first basement of the first building 21. The irradiation room 25 to install the slit-type irradiation device 51 is built on the first floor of the second building 22. That is, the level of the accelerator room 24 differs from the level of the irradiation room 25, and therefore the floor F of the accelerator room 24 differs in height from the floor F of the irradiation room 25.

For example, the floor F of the accelerator room 24 is set to a lower position than the floor F of the irradiation room 25. That is, the slit-type irradiation device 51 is installed at a higher position than the circular accelerator 4.

The main transport line 5 extends from the accelerator room 24 in the first building 21 to the underground channel 29 in the second building 22 through the connecting passage 23.

The sub-transport line 6 is bent obliquely upwards from the underground channel 29 and extended to the irradiation room 25 on the first floor of the second building 22. The trajectory of charged particle beams is bent obliquely upwards by the deflection electromagnets 14 of the sub-transport line 6.

In the tilted position of the slit-type irradiation device 51, the floor F of the irradiation room 25 is at a higher position than the floor F of the accelerator room 24. This may reduce the number of deflection electromagnets 14 in the sub-transport line 6 when charged particle beams enter the slit-type irradiation device 51 from the sub-transport line 6 in an oblique direction (or a vertical direction).

In this connection, the fourth embodiment is designed to tilt the upper part of the main body 54 of the slit-type irradiation device 51 to be separated from the patient P, whereas the upper part of the main body 54 can be tilted toward the patient P. In addition, it is possible to use the slit-type irradiation device 51 without any tilt.

### Fifth Embodiment

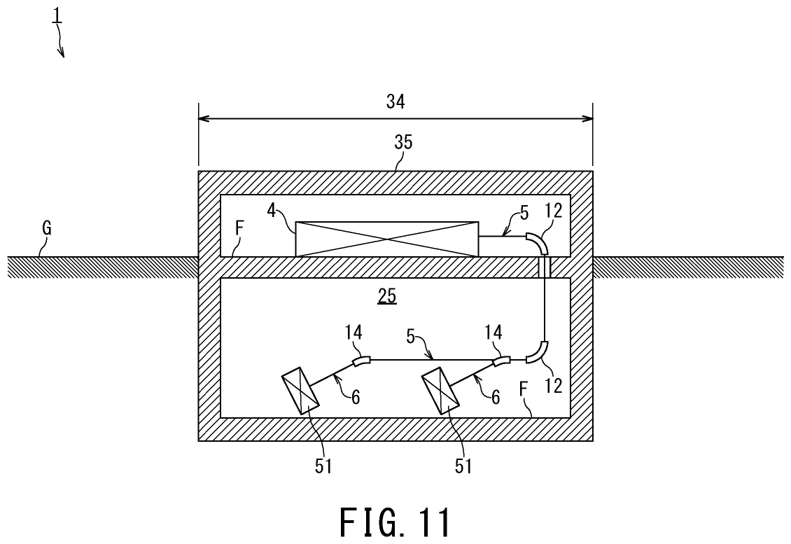

Next, the fifth embodiment will be described with reference to FIG. 11. In this connection, the same constituent parts as the constituent parts of the foregoing embodiments will be denoted by the same reference signs; hence, duplication descriptions thereof will be mitted here.

In the fifth embodiment, the slit-type irradiation device 51 exemplifies an irradiation device. In addition, a single building 35 is built on a single building site 34. The building 35 has a first floor above ground and a first basement below ground.

The accelerator room 24 to install machinery such as the circular accelerator 4 is built on the first floor of the building 35. The irradiation room 25 to install the slit-type irradiation device 51 is built in the first basement of the building 35. That is, the level of the accelerator room 24 differs from the level of the irradiation room 25, and therefore the floor F of the accelerator room 24 differs in height from the floor F of the irradiation room 25.

The main transport line 5 extends from the accelerator room 24 of the building 35 above ground to the irradiation room 25 below ground. Charged particle beams are transported to the irradiation room 25 according to the trajectory being bent by the deflection electromagnets 12 of the main transport line 5.

The main transport line 5 extends to the neighborhood of the slit-type irradiation device 51 and further extends in the horizontal direction at a higher position than the slit-type irradiation device 51. The sub-transport line 6 extends obliquely downwards from the main transport line 5 and connects to the slit-type irradiation device 51. The trajectory of charged particle beams is bent downwards by the deflection electromagnets 14 of the sub-transport line 6.

In tilted position of the slit-type irradiation device 51, the slit-type irradiation device 51 is installed at a lower position than the main transport line 5. This may reduce the number of deflection electromagnets 14 in the sub-transport line 6 when charged particle beams enter the slit-type irradiation device 51 from the sub-transport line 6 in an oblique direction (or a vertical direction).

Heretofore, the present invention has been described with reference to the first embodiment through the fifth embodiment, whereas any configuration employed in any embodiment can be applied to other embodiments, alternatively, it is possible to combine various configurations applied to the foregoing embodiments.

In the foregoing embodiments, the patient P who is a human exemplifies an irradiation target receiving charged particle beams, but other types of targets can be used. For example, it is possible to designate another animal such as a dog and a cat as an irradiation target. That is, the particle beam irradiation system 1 can be used to conduct radiation treatment on these animals.

According to at least one embodiment described above, which is designed to include the first building 21 and the second building 22, it is possible to install the particle beam irradiation system 1 efficiently on a limited site in an urban area or the like, thus contributing to a reduction of construction costs.

Various embodiments have been described above with respect to the present invention, whereas these embodiments are illustrative and do not intend to limit the scope of the invention. These embodiments can be realized in other various forms; hence, it is possible to introduce various omissions, substitutions, modifications, and combinations without departing from the gist of the invention. These embodiments and modifications shall fall within the scope and the gist of the invention and within the invention as defined in the claims and equivalents thereof.

1. A particle beam irradiation system comprising:
a first building;
a second building different from the first building;
an accelerator installed in the first building configured to accelerate a charged particle beam;
a beam transport line configured to transport the charged particle beam accelerated by the accelerator; and
an irradiation device installed in the second building configured to irradiate an irradiation target with the charged particle beam transported by the beam transport line.

2. The particle beam irradiation system according to claim 1, wherein the first building is built on one site of two building sites adjoining together across a road site, while the second building is built on another site of the two building sites.

3. The particle beam irradiation system according to claim 1, wherein the beam transport line has a linear portion for straightly connecting the first building and the second building.

4. The particle beam irradiation system according to claim 2, wherein the beam transport line has a linear portion for straightly connecting the first building and the second building.

5. The particle beam irradiation system according to claim 1, wherein the beam transport line has a connecting portion underground, which connects the first building and the second building.

6. The particle beam irradiation system according to claim 2, wherein the beam transport line has a connecting portion underground, which connects the first building and the second building.

7. The particle beam irradiation system according to claim 1, wherein a floor of an accelerator room configured to install the accelerator therein differs in height from a floor of an irradiation room configured to install the irradiation device therein.

8. The particle beam irradiation system according to claim 2, wherein a floor of an accelerator room configured to install the accelerator therein differs in height from a floor of an irradiation room configured to install the irradiation device therein.

9. The particle beam irradiation system according to claim 1, wherein an accelerator room configured to install the accelerator therein differs in level from an irradiation room configured to install the irradiation device therein.

10. The particle beam irradiation system according to claim 2, wherein an accelerator room configured to install the accelerator therein differs in level from an irradiation room configured to install the irradiation device therein.

11. The particle beam irradiation system according to claim 1, wherein the irradiation device is a stationary irradiation device having a fixed angle of emitting the charged particle beam toward an isocenter to place the irradiation target, thus emitting the charged particle beam at another angle than a horizontal direction.

12. The particle beam irradiation system according to claim 2, wherein the irradiation device is a stationary irradiation device having a fixed angle of emitting the charged particle beam toward an isocenter to place the irradiation target, thus emitting the charged particle beam at another angle than a horizontal direction.

13. The particle beam irradiation system according to claim 1, wherein the irradiation device is equipped with an irradiation nozzle configured to change an irradiation direction of the charged particle beam toward an isocenter, wherein the irradiation nozzle is subject to movement around the isocenter to place the irradiation target along an entire circumference at equal distances apart from the isocenter.

14. The particle beam irradiation system according to claim 2, wherein the irradiation device is equipped with an irradiation nozzle configured to change an irradiation direction of the charged particle beam toward an isocenter, wherein the irradiation nozzle is subject to movement around the isocenter to place the irradiation target along an entire circumference at equal distances apart from the isocenter.

15. The particle beam irradiation system according to claim 1, wherein the irradiation device is equipped with an irradiation nozzle configured to change an irradiation direction of the charged particle beam toward an isocenter, wherein the irradiation nozzle is subject to movement around the isocenter to place the irradiation target in a circumferential direction at equal distances apart from the isocenter, and wherein the movement of the irradiation nozzle is equal to 240 degrees or less.

16. The particle beam irradiation system according to claim 2, wherein the irradiation device is equipped with an irradiation nozzle configured to change an irradiation direction of the charged particle beam toward an isocenter, wherein the irradiation nozzle is subject to movement around the isocenter to place the irradiation target in a circumferential direction at equal distances apart from the isocenter, and wherein the movement of the irradiation nozzle is equal to 240 degrees or less.

17. The particle beam irradiation system according to claim 1, wherein the irradiation device is a slit-type irradiation device having a slit formed to extend in a circumferential direction around an isocenter to place the irradiation target, thus emitting the charged particle beam toward the isocenter at an arbitrary angle.

18. The particle beam irradiation system according to claim 2, wherein the irradiation device is a slit-type irradiation device having a slit formed to extend in a circumferential direction around an isocenter to place the irradiation target, thus emitting the charged particle beam toward the isocenter at an arbitrary angle.

19. The particle beam irradiation system according to claim 17, wherein the irradiation device is installed in a tilted position such that a reference trajectory of the charged particle beam undeflected by the irradiation device is tilted against a horizontal direction.

20. The particle beam irradiation system according to claim 18, wherein the irradiation device is installed in a tilted position such that a reference trajectory of the charged particle beam undeflected by the irradiation device is tilted against a horizontal direction.
